(19) United States
(12) Patent Application Publication
(10) Pub. No.: US 2026/0345497 A1
(43) Pub. Date: Nov. 5, 2026

(54) PHARMACEUTICAL COMPOSITIONS

(71) Applicant: ASTRAZENECA AB, Södertälje (SE)

(72) Inventors: Farhan Abdel Karim Mohammad AL HUSBAN, Cambridge (GB); Tomos Rees MORGAN, Cambridge (GB)

(21) Appl. No.: 19/487,550

(22) PCT Filed: May 24, 2024
(86) PCT No.: PCT/EP2024/064426

§ 371 Date: Nov. 25, 2025

(30) Foreign Application Priority Data
May 26, 2023 (EP) 23175788.1

(51) Int. Cl. A61K 9/20; A61K 9/28; A61K 31/519

(52) U.S. Cl. CPC A61K 9/2054; A61K 9/2009; A61K 9/2013; A61K 9/2813; A61K 9/2826; A61K 9/284; A61K 9/2866; A61K 31/519

(57) ABSTRACT

The present specification relates to pharmaceutical compositions comprising Compound [I] ((S)-4-amino-N-(1-(4-chlorophenyl)-3-hydroxypropyl)-1-(7H-pyrrolo[2,3-d]pyrimidin-4-yl)piperidine-4-carboxamide), microcrystalline cellulose (MCC) and dicalcium phosphate anhydrous (DCPA), for example tablets with immediate release properties.

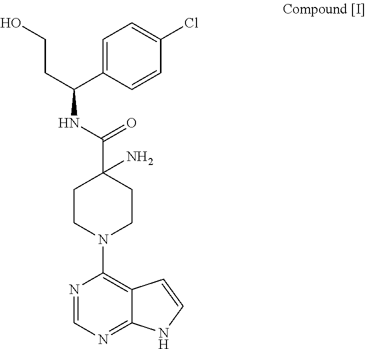 [SMILES: NC1(C(=O)N[C@@H](CCO)c2ccc(Cl)cc2)CCN(c2ncnc3[nH]ccc23)CC1]

### CROSS-REFERENCE TO RELATED APPLICATIONS

This application claims priority to European Patent Application No. 23175788.1, filed 26 May 2023, the disclosure of which is incorporated by reference herein.

### FIELD

This specification relates to pharmaceutical compositions comprising the selective AKT inhibitor (S)-4-amino-N-(1-(4-chlorophenyl)-3-hydroxypropyl)-1-(7H-pyrrolo[2,3-d]pyrimidin-4-yl)piperidine-4-carboxamide (Compound [I]) or a pharmaceutically acceptable salt thereof, and certain pharmaceutically acceptable excipients. Compound [I] is also known as capivasertib and AZD5363, and is disclosed in WO2009/047563 as Example 9.

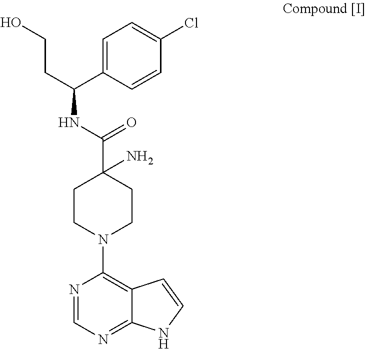 [SMILES: NC1(C(=O)N[C@@H](CCO)c2ccc(Cl)cc2)CCN(c2ncnc3[nH]ccc23)CC1]

The specification further relates to oral solid dosage forms, for example tablets, comprising Compound [I] or a pharmaceutically acceptable salt thereof and certain pharmaceutically acceptable excipients. The pharmaceutical compositions and oral solid dosage forms according to the specification have advantageous properties that facilitate for large scale manufacture as well as exhibiting good storage stability and other useful characteristics. This facilitates their use in methods of treatment, for example methods for treating cancer.

### BACKGROUND

AKT is part of the cAMP-dependent protein kinases A, cGMP-dependent protein kinases G, and phospholipid-dependent protein kinases C (AGC) families. Mammalian cells express three closely related AKT isoforms: AKT1 (protein kinase Bα [PKBα]), AKT2 (PKBβ) and AKT3 (PKBγ), all encoded by different genes. AKT is a node of multiple signalling pathways promoting tumourigenesis, inhibiting apoptosis, impacting on the cell cycle, and promoting invasion and migration. The phosphoinositide 3-kinase/protein kinase/phosphatase and tensin homologue (PI3K/AKT/PTEN) pathway regulates cell growth, proliferation and survival (Brown J S, Banerji U. Pharmacol Ther 2017; 172:101-15) and is frequently deregulated in cancer.

Capivasertib (Compound [I]) is a potent, selective inhibitor of the kinase activity of all three AKT isoforms. It is being developed as a potential treatment for solid and haematological malignancies (see trials referring to capivasertib, AZD5363 and/or (S)-4-amino-N-(1-(4-chlorophenyl)-3-hydroxypropyl)-1-(7H-pyrrolo[2,3-d]pyrimidin-4-yl)piperidine-4-carboxamide on clinicaltrials.gov [for example trials NCT03997123, NCT04305496, NCT04862663, NCT05348577 or NCT04493853] nih.gov, clinicaltrialsregister.eu or similar public clinical trial repositories).

The high drug loading required to achieve the required doses of capivasertib in tablets with globally acceptable sizes and shapes requires careful formulation to offset exceptionally cohesive and adhesive properties of the drug substance (cf. Example 4). The “stickiness” of the active pharmaceutical ingredient set it apart from other drug substances and means that applying known formulations to capivasertib may be difficult, meaning there is a need for new formulations that are suitable for therapy as well as commercial manufacture.

To address this need the present specification discloses pharmaceutical compositions of capivasertib comprising a combination of microcrystalline cellulose (MCC) and dicalcium phosphate anhydrous (DCPA, also known as anhydrous calcium hydrogen phosphate). Not only have these compositions been found to be suitable for oral dosing of patients (for example providing immediate release of the drug), but their physical properties (including tensile strength, strain rate sensitivity and ability to aid deformation compression) confer significant advantages in terms of improved tablet manufacturability, for instance with respect to powder flow to the roller compactor after dry granulation, punch filming and tablet appearance after coating (cf. Examples 1-6).

### SUMMARY

In a first aspect the present specification provides a pharmaceutical composition comprising Compound [I] or a pharmaceutically acceptable salt thereof, microcrystalline cellulose and dicalcium phosphate anhydrous. The pharmaceutical compositions according to the specification may comprise further excipients, for example disintegrants and/or lubricants.

In a further aspect the specification provides an oral solid dosage form comprising Compound [I] or a pharmaceutically acceptable salt thereof, microcrystalline cellulose and dicalcium phosphate anhydrous. The oral solid dosage forms according to this aspect may comprise further excipients, for example disintegrants or lubricants and may be provided as a capsule or tablet.

In a further aspect the specification provides a method for producing a tablet according to the specification comprising the steps of i) Blending a mixture comprising Compound [I] or a pharmaceutically acceptable salt thereof, microcrystalline cellulose, and dicalcium phosphate anhydrous and optionally a disintegrant and/or a lubricant to form a blend; ii) Dry granulating the blend; and iii) Compressing the dry granulated blend into a tablet.

In a further aspect the specification provides a pharmaceutical composition comprising Compound [I] or a pharmaceutically acceptable salt thereof, microcrystalline cellulose and dicalcium phosphate anhydrous, or an oral solid dosage form comprising such a pharmaceutical composition, for use in therapy, for example in the treatment of cancer.

In a further aspect the specification provides the use of a pharmaceutical composition comprising Compound [I] or a pharmaceutically acceptable salt thereof, microcrystalline cellulose and dicalcium phosphate anhydrous, or an oral solid dosage form comprising such a pharmaceutical composition, for the preparation of a medicament for use in the treatment of cancer.

In a further aspect the specification provides a method of treating cancer in a patient in need of such treatment, comprising administering to the patient a therapeutically effective amount of a pharmaceutical composition comprising Compound [I] or a pharmaceutically acceptable salt thereof, microcrystalline cellulose and dicalcium phosphate anhydrous, or an oral solid dosage form comprising such a pharmaceutical composition.

Further aspects of the disclosure will be apparent to one skilled in the art from reading this specification.

### FIGURES

Reference is made to the following FIGURES:

FIG. 1: Disintegration times for formulations Examples 1e-1h when compressed into tablets at varying pressure.

### DETAILED DESCRIPTION

The invention detailed in this specification should not be interpreted as being limited to any of the individual recited embodiments. Other embodiments will be readily apparent to a reader skilled in the art.

All % weights (w/w) throughout this specification are expressed in relation to the total weight of the pharmaceutical composition, unless indicated otherwise.

Ratios described throughout this specification are weight ratios of the specified components, unless indicated otherwise.

### Pharmaceutical Compositions

As noted above, the present specification provides a pharmaceutical composition comprising Compound [I] or a pharmaceutically acceptable salt thereof, microcrystalline cellulose and dicalcium phosphate anhydrous.

The compositions according to the specification possess various advantageous properties that render them useful in the field of pharmaceuticals. For example, the good flow properties of the blend of Compound [I] and the specified excipients allow for efficient processing for the manufacture of oral solid dosage forms, for example via dry granulation. The oral solid dosage forms, for example tablets formed from the compositions according to the specification, possess good stability, immediate release properties and exhibit excellent structural integrity. They also have favourable tensile strength, strain rate sensitivity and deformation compression aid properties which lead to significant advantages in terms of improved tablet manufacturability, for example with respect to ease of roller compaction, punch filming and appearance after coating.

In one embodiment there is provided a pharmaceutical composition comprising Compound [I] or a pharmaceutically acceptable salt thereof, microcrystalline cellulose and dicalcium phosphate anhydrous.

In embodiments there is provided a pharmaceutical composition comprising Compound [I], microcrystalline cellulose and dicalcium phosphate anhydrous.

In embodiments Compound [I] is present as crystalline Form B. Crystalline Form B is disclosed in WO2013/156772.

In embodiments there is provided a pharmaceutical composition comprising a pharmaceutically acceptable salt of Compound [I], microcrystalline cellulose and dicalcium phosphate anhydrous.

In embodiments where a w/w % of a pharmaceutically acceptable salt of Compound [I] is mentioned, the w/w % is calculated with reference to the weight of an equivalent amount of Compound [I].

The term “pharmaceutically acceptable” is used to specify that an object (for example a salt or excipient) is suitable for use in patients. An example list of pharmaceutically acceptable salts can be found in the “Handbook of Pharmaceutical Salts: Properties, Selection and Use”, P. H. Stahl and C. G. Wermuth, editors, Weinheim/Zurich:Wiley-VCH/VFiCA, 2002, or earlier/subsequent editions.

Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, p-toluene sulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminium. Organic bases from which salts can be derived include, for example, primary, secondary, and tertiary amines, substituted amines including naturally occurring substituted amines, cyclic amines and basic ion exchange resins. Examples include isopropylamine, trimethylamine, diethylamine, triethylamine, tripropylamine, and ethanolamine.

In embodiments the pharmaceutical composition comprises up to 65% w/w of Compound [I] or a pharmaceutical salt thereof.

In embodiments the pharmaceutical composition comprises up to 65% w/w of Compound [I].

In embodiments the pharmaceutical composition comprises 40%-50% w/w of Compound [I] or a pharmaceutically acceptable salt thereof.

In embodiments the pharmaceutical composition comprises 40%-50% w/w of Compound [I].

In embodiments the pharmaceutical composition comprises about 50% w/w of Compound [I] or a pharmaceutically acceptable salt thereof.

In embodiments the pharmaceutical composition comprises about 50% w/w of Compound [I].

In embodiments the pharmaceutical composition comprises 50% w/w of Compound [I] or a pharmaceutically acceptable salt thereof.

In embodiments the pharmaceutical composition comprises 50% w/w of Compound [I].

In embodiments the pharmaceutical composition comprises about 40% w/w of Compound [I] or a pharmaceutically acceptable salt thereof.

In embodiments the pharmaceutical composition comprises about 40% w/w of Compound [I].

In embodiments the pharmaceutical composition comprises 40% w/w of Compound [I] or a pharmaceutically acceptable salt thereof.

In embodiments the pharmaceutical composition comprises 40% w/w of Compound [I].

In embodiments the pharmaceutical composition comprises about 160 mg of Compound [I].

In embodiments the pharmaceutical composition comprises 160 mg of Compound [I].

In embodiments the pharmaceutical composition comprises about 200 mg of Compound [I].

In embodiments the pharmaceutical composition comprises 200 mg of Compound [I].

In embodiments the ratio of microcrystalline cellulose to dicalcium phosphate anhydrous is from 45:55 to 70:30.

In embodiments the ratio of microcrystalline cellulose to dicalcium phosphate anhydrous is from 55:45 to 68:32.

In embodiments the ratio of microcrystalline cellulose to dicalcium phosphate anhydrous is about 6:4.

In embodiments the ratio of microcrystalline cellulose to dicalcium phosphate anhydrous is 6:4.

As the Examples show, these ratios of microcrystalline cellulose to dicalcium phosphate anhydrous are those in which the pharmaceutical compositions a) exhibit a strain rate sensitivity below around 20% that allows high speed processing, for example high speed processing by dry granulation, and b) that can be processed to deliver tablets with, among other advantages, consistently high tensile strengths (>2 MPa).

In embodiments the pharmaceutical composition comprises 40%-50% w/w of Compound [I] or a pharmaceutically acceptable salt thereof, and the ratio of microcrystalline cellulose to dicalcium phosphate anhydrous is from 45:55 to 70:30.

In embodiments the pharmaceutical composition comprises 40%-50% w/w of Compound [I], and the ratio of microcrystalline cellulose to dicalcium phosphate anhydrous is from 45:55 to 70:30.

In embodiments the pharmaceutical composition comprises 40%-50% w/w of Compound [I] or a pharmaceutically acceptable salt thereof, and the ratio of microcrystalline cellulose to dicalcium phosphate anhydrous is from 55:45 to 68:32.

In embodiments the pharmaceutical composition comprises 40%-50% w/w of Compound [I], and the ratio of microcrystalline cellulose to dicalcium phosphate anhydrous is from 55:45 to 68:32.

In embodiments the pharmaceutical composition comprises about 50% w/w of Compound [I] or a pharmaceutically acceptable salt thereof, and the ratio of microcrystalline cellulose to dicalcium phosphate anhydrous is from 55:45 to 68:32.

In embodiments the pharmaceutical composition comprises about 50% w/w of Compound [I], and the ratio of microcrystalline cellulose to dicalcium phosphate anhydrous is from 55:45 to 68:32.

In embodiments the pharmaceutical composition comprises about 50% w/w of Compound [I], and the ratio of microcrystalline cellulose to dicalcium phosphate anhydrous is about 6:4.

In embodiments the pharmaceutical composition comprises about 40% w/w of Compound [I] or a pharmaceutically acceptable salt thereof, and the ratio of microcrystalline cellulose to dicalcium phosphate anhydrous is from 45:55 to 70:30.

In embodiments the pharmaceutical composition comprises about 40% w/w of Compound [I], and the ratio of microcrystalline cellulose to dicalcium phosphate anhydrous is from 45:55 to 70:30.

In embodiments the pharmaceutical composition comprises about 40% w/w of Compound [I], and the ratio of microcrystalline cellulose to dicalcium phosphate anhydrous is about 6:4.

In embodiments the combined amount of microcrystalline cellulose and dicalcium phosphate anhydrous in the pharmaceutical composition is from 15% w/w to 65% w/w.

In embodiments the combined amount of microcrystalline cellulose and dicalcium phosphate anhydrous in the pharmaceutical composition is from 30% w/w to 65% w/w.

In embodiments the combined amount of microcrystalline cellulose and dicalcium phosphate anhydrous in the pharmaceutical composition is from 40% w/w to 60% w/w.

In embodiments the combined amount of microcrystalline cellulose and dicalcium phosphate anhydrous in the pharmaceutical composition is from 50% w/w to 60% w/w.

In embodiments the combined amount of microcrystalline cellulose and dicalcium phosphate anhydrous in the pharmaceutical composition is from 50% w/w to 55% w/w.

In embodiments the combined amount of microcrystalline cellulose and dicalcium phosphate anhydrous in the pharmaceutical composition is 53-54% w/w.

In embodiments the combined amount of microcrystalline cellulose and dicalcium phosphate anhydrous in the pharmaceutical composition is about 53.5% w/w.

In embodiments the pharmaceutical composition further comprises at least one disintegrant in an amount of up to 10% w/w.

Use of a disintegrant in the pharmaceutical compositions according to the specification helps promote disintegration of the composition on contact with a fluid such as water.

In embodiments the pharmaceutical composition further comprises at least one disintegrant in an amount of up to 5% w/w.

In embodiments the pharmaceutical composition further comprises at least one disintegrant in an amount of about 5% w/w.

In embodiments the at least one disintegrant is croscarmellose sodium.

In embodiments the pharmaceutical composition further comprises at least one lubricant in an amount of up to 4% w/w.

Use of a lubricant in the pharmaceutical compositions according to the specification helps ensure that the production of tablets is efficient and is associated with advantageously low ejection forces from tableting presses and minimisation of flaws in the product tablets.

In embodiments the pharmaceutical composition further comprises at least one lubricant in an amount of up to 1.5% w/w.

In embodiments the pharmaceutical composition further comprises at least one lubricant in an amount of about 1.5% w/w.

In embodiments the at least one lubricant is magnesium stearate.

In one embodiment is provided a pharmaceutical composition comprising:
Compound [I];
Microcrystalline cellulose and dicalcium phosphate anhydrous, where the ratio of microcrystalline cellulose to dicalcium phosphate anhydrous is about 6:4;
A disintegrant; and
A lubricant.

In one embodiment is provided a pharmaceutical composition comprising:
40%-50% w/w Compound [I]; and
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 40%-60% w/w.

In one embodiment is provided a pharmaceutical composition comprising:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 50%-55% w/w;
Croscarmellose sodium; and
Magnesium stearate.

In one embodiment is provided a pharmaceutical composition comprising:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 50%-55% w/w;
Croscarmellose sodium in an amount of about 5% w/w; and
Magnesium stearate in an amount of about 1.5% w/w.

In one embodiment is provided a pharmaceutical composition comprising:
40% w/w Compound [I];
microcrystalline cellulose and dicalcium phosphate anhydrous, where the ratio of microcrystalline cellulose to dicalcium phosphate anhydrous is 6:4, and the combined amount of microcrystalline cellulose and dicalcium phosphate anhydrous is 53.5% w/w;
Croscarmellose sodium in an amount of 5% w/w; and
Magnesium stearate in an amount of 1.5% w/w.

In one embodiment there is provided a pharmaceutical composition selected from Example 1a, Example 1b, Example 2, Examples 3a-3f, Example 5a and Example 5b.

In one embodiment there is provided any pharmaceutical composition disclosed in the Example section.

### Oral Solid Dosage Forms

In one embodiment there is provided an oral solid dosage form comprising any of the pharmaceutical compositions disclosed in this specification.

In some embodiments the oral solid dosage form is an immediate release oral solid dosage form.

“Immediate release” is used in its conventional sense to refer to a dosage form that provides for release of Compound [I] immediately after administration. For example, an immediate release pharmaceutical composition means a composition in which the dissolution rate of the drug from the composition is 80% or more after 30 minutes from the beginning a dissolution test, which is carried out in accordance with a dissolution test (paddle method) described in the United States Pharmacopoeia under the conditions that 900 mL of an appropriate test fluid (such as a USP buffer, pH 6.8 or pH 7.4) is used and the paddle rotation speed is 50, 75 or 100 rpm.

In some embodiments the oral solid dosage form is a capsule or a tablet.

In some embodiments the oral solid dosage form is a capsule.

In some embodiments the oral solid dosage form is a capsule containing a pharmaceutical composition disclosed herein.

In some embodiments the oral solid dosage form is a tablet.

In some embodiments the tablet comprises a core and a coating over the core.

In some embodiments the core comprises a pharmaceutical composition disclosed herein.

In some embodiments the core consists of a pharmaceutical composition disclosed herein.

Coating can advantageously further increase the shelf life of the tablets as the coating can protect tablets from light, moisture and oxidation. Coatings may also be used to improve the aesthetics of the tablet, as well as improving tablet mechanical strength and to mask odours or taste.

Polymers used in the coating can be chosen, for example, from a cellulosic polymer such as hydroxypropyl methyl cellulose (HPMC) as found in Opadry® I (www.colorcon.com) and the Aquarius coating systems (www.ashland.com), hydroxypropyl cellulose (HPC) and ethyl cellulose (EC) or vinyls such as polyvinyl alcohol. Plasticizing agents are used to improve elasticity of the coating film and reduce the film forming temperature of the polymer thus allowing lower temperature processing. Suitable plasticizing agents include propylene glycol or polyethylene glycol or glycerol, acetate esters such as triacetin (glycerol triacetate) or triethyl citrate (TEC), glycerides such as acetylated monoglycerides, and mineral and vegetable oils. Colourants and pigments are used to increase the opacity and/or light protection of the film and provide colouration. Suitable colourants include indigo carmine, tartrazine, allura red, and quinoline yellow; inorganic pigments such as titanium dioxide, iron oxides, and pearlescent pigments and natural pigments such as vegetable juice, carotenoids, and turmeric.

The coating may also incorporate further functional ingredients such as glidants, flavours and viscosity modifiers all of which are well known in the art. General details on pharmaceutical coatings can be found in Aulton's Pharmaceutics, 5^{th} Edition, 2018, Elsevier, at e.g. page 580-596.

In embodiments the coating is a film coating.

In embodiments the coating is an immediate release film coating.

In embodiments the coating comprises hypromellose 2910, copovidone plasdone S630, polyethylene glycol 3350, polydextrose, at least one caprylic/capric triglyceride, and optionally at least one opacifier (for example titanium dioxide) and/or at least one colouring agent (for example an iron oxide colourant, such as iron oxide red, iron oxide yellow, or iron oxide black).

In embodiments the coating comprises hypromellose 2910, copovidone plasdone S630, polyethylene glycol 3350, polydextrose, at least one caprylic/capric triglyceride, titanium dioxide, iron oxide yellow, iron oxide red and iron oxide black.

In embodiments the coating comprises hypromellose 2910, copovidone plasdone S630, polyethylene glycol 3350, polydextrose, at least one caprylic/capric triglyceride, titanium dioxide, iron oxide yellow, iron oxide red and iron oxide black in the w/w quantities expressed in Table 14 or Table 16.

In embodiments the coating is Aquarius® preferred beige (BPP315509).

In one embodiment is provided an oral solid dosage form which is a tablet comprising a core and a optionally a coating over the core, wherein the core comprises:
Compound [I];
Microcrystalline cellulose and dicalcium phosphate anhydrous, where the ratio of microcrystalline cellulose to dicalcium phosphate anhydrous is about 6:4;
A disintegrant; and
A lubricant.

In one embodiment is provided an oral solid dosage form which is a tablet comprising a core and a optionally a coating over the core, wherein the core comprises:
Compound [I];
Microcrystalline cellulose and dicalcium phosphate anhydrous, where the ratio of microcrystalline cellulose to dicalcium phosphate anhydrous is about 6:4;
Croscarmellose sodium; and
Magnesium stearate.

In one embodiment is provided an oral solid dosage form which is a tablet comprising a core and a optionally a coating over the core, wherein the core comprises:
40%-50% w/w Compound [I]; and
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 40%-60% w/w.

In one embodiment is provided an oral solid dosage form which is a tablet comprising a core and a optionally a coating over the core, wherein the core comprises:
40%-50% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 40%-60% w/w;
Croscarmellose sodium in an amount of about 5% w/w; and
Magnesium stearate in an amount of about 1.5% w/w.

In one embodiment is provided an oral solid dosage form which is a tablet comprising a core and a optionally a coating over the core, wherein the core comprises:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 50%-55% w/w;
Croscarmellose sodium in an amount of about 5% w/w; and
Magnesium stearate in an amount of about 1.5% w/w.

In one embodiment is provided an oral solid dosage form which is a tablet comprising a core and a optionally a coating over the core, wherein the core comprises:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is about 53.5% w/w;
Croscarmellose sodium in an amount of about 5% w/w; and
Magnesium stearate in an amount of about 1.5% w/w.

In embodiments where a w/w % of a component of the core (for example Compound [I], microcrystalline cellulose, dicalcium phosphate anhydrous, croscarmellose sodium, and/or magnesium stearate) is mentioned, the w/w % is calculated with reference to total weight of the core.

The oral solid dosage forms according to the specification comprise Compound [I] in an amount suitable for dosing to a patient in need thereof, either as a single dosage form or as a plurality of dosage forms. The dose of Compound [I] required for the therapeutic or prophylactic treatment of a particular disease or medical condition will necessarily be varied depending on, for example, the host treated and the severity of the illness being treated. The amount of the active compound administered will be dependent on the subject being treated, the severity of the disorder or condition, the rate of administration, the disposition of the compound and the discretion of the prescribing physician.

In embodiments the oral solid dosage form contains 80 mg, 160 mg or 200 mg of Compound [I] or a pharmaceutically acceptable salt thereof.

In embodiments the oral solid dosage form contains 80 mg, 160 mg or 200 mg of Compound [I].

In embodiments the oral solid dosage form contains 160 mg of Compound [I].

In embodiments the oral solid dosage form contains 200 mg of Compound [I].

In one embodiment there is provided an oral solid dosage form comprising:
200 mg Compound [I];
160.5 mg microcrystalline cellulose;
107 mg dicalcium phosphate anhydrous;
25 mg croscarmellose sodium; and
7.5 mg magnesium stearate.

In one embodiment there is provided an oral solid dosage form which is a tablet comprising a core and a coating over the core, wherein the core comprises:
200 mg Compound [I];
160.5 mg microcrystalline cellulose;
107 mg dicalcium phosphate anhydrous;
25 mg croscarmellose sodium; and
7.5 mg magnesium stearate.

In one embodiment there is provided an oral solid dosage form which is a tablet comprising a core and a coating over the core, wherein the core comprises:
200 mg Compound [I];
160.5 mg microcrystalline cellulose;
107 mg dicalcium phosphate anhydrous;
25 mg croscarmellose sodium; and
7.5 mg magnesium stearate;
   and the coating comprises hypromellose 2910, copovidone plasdone S630, polyethylene glycol 3350, polydextrose, at least one caprylic/capric triglyceride, and optionally at least one opacifier (for example titanium dioxide) and/or at least one colouring agent (for example an iron oxide colourant, such as iron oxide red, iron oxide yellow, or iron oxide black).

In one embodiment there is provided an oral solid dosage form, comprising:
160 mg Compound [I];
128.4 mg microcrystalline cellulose;
85.6 mg dicalcium phosphate anhydrous;
20 mg croscarmellose sodium; and
6 mg magnesium stearate.

In one embodiment there is provided an oral solid dosage form which is a tablet comprising a core and a coating over the core, wherein the core comprises:
160 mg Compound [I];
128.4 mg microcrystalline cellulose;
85.6 mg dicalcium phosphate anhydrous;
20 mg croscarmellose sodium; and
6 mg magnesium stearate.

In one embodiment there is provided an oral solid dosage form which is a tablet comprising a core and a coating over the core, wherein the core comprises:
160 mg Compound [I];
128.4 mg microcrystalline cellulose;
85.6 mg dicalcium phosphate anhydrous;
20 mg croscarmellose sodium; and
6 mg magnesium stearate;
   and the coating comprises hypromellose 2910, copovidone plasdone S630, polyethylene glycol 3350, polydextrose, at least one caprylic/capric triglyceride, and optionally at least one opacifier (for example titanium dioxide) and/or at least one colouring agent (for example an iron oxide colourant, such as iron oxide red, iron oxide yellow, or iron oxide black).

In one embodiment there is provided an oral solid dosage form which is a tablet comprising a core consisting of the core components listed in Table 16 (i.e. Example 6a or Example 6b, optionally including relative amounts).

In one embodiment there is provided an oral solid dosage form which is a tablet comprising a core consisting of the core components listed in Table 16 (i.e. Example 6a or Example 6b, optionally including relative amounts), and a coating over the core, wherein the coating consists of the coating components listed in Table 16 (i.e. Example 6a or Example 6b, optionally including relative amounts).

### Pharmaceutical Composition Manufacture

In one embodiment, a pharmaceutical composition as described herein is a dry granulated pharmaceutical composition.

In one embodiment, the dry granulated formulation is obtainable by a process comprising the steps of: (a) blending Compound [I] or a pharmaceutically acceptable salt thereof, microcrystalline cellulose, and dicalcium phosphate anhydrous and optionally a disintegrant and/or a lubricant to form a blend; (b) dry granulating the blend and optionally blending in a lubricant.

In one embodiment, the dry granulated formulation is obtainable by a process comprising the steps of: (a) blending Compound [I] or a pharmaceutically acceptable salt thereof, microcrystalline cellulose, dicalcium phosphate anhydrous, a disintegrant and a lubricant to form a blend; (b) dry granulating the blend and optionally blending in further lubricant.

In one embodiment, the dry granulation process in step (b) is performed by roller compaction of the blend followed by milling.

### Tablet Manufacture Processes

A typical tableting process commences by introducing, in batches or continuously, a bulk powder mix or granules into a feeder frame that fills a tabletting die with a predetermined weight of material in a consistent manner. The contents of the filled tabletting die are then compressed, typically by the action of the upper and lower punches to form a compacted formulation that is then ejected to form intact tablets.

Tablets prepared from the pharmaceutical compositions according to the specification have advantageously high tensile strength and consequently exhibit good mechanical stability. The compositions according to the specification comprise a blend of the active pharmaceutical ingredient (API), Compound [I], with MCC and DCPA that displays good strain rate sensitivity (SRS), of ca 20% or less, and this low strain rate sensitivity allows for rapid blending of the API and the excipients in the production of tablets containing high w/w amounts of Compound [I]. The compositions according to the specification deliver tablets that can be reproducibly manufactured without over-compression that have a porosity value commensurate with reproducible dissolution profile.

Tablets according to the specification are those prepared from the compositions according to the specification via standard techniques including roller compaction or direct compaction.

In one embodiment there is provided a process for manufacturing an oral solid dosage form which is a tablet, comprising the steps of:
i) Blending a mixture comprising Compound [I] or a pharmaceutically acceptable salt thereof, microcrystalline cellulose, and dicalcium phosphate anhydrous and optionally a disintegrant and/or a lubricant to form a blend;
ii) Dry granulating the blend; and
iii) Compressing the dry granulated blend into a tablet.

In some embodiments the dry granulation process in step ii) is performed by roller compaction of the blend followed by milling.

In one embodiment there is provided a process for manufacturing an oral solid dosage form which is a tablet, comprising the steps of:
i) Blending a mixture comprising Compound [I] or a pharmaceutically acceptable salt thereof, microcrystalline cellulose, and dicalcium phosphate anhydrous, a disintegrant and a lubricant to form a blend;
ii) Dry granulating the blend;
iii) Blending further lubricant into the dry granulated blend; and
iv) Compressing the blend of step iii) into a tablet.

In some embodiments the dry granulation process in step ii) is performed by roller compaction of the blend followed by milling.

In one embodiment there is provided a process for manufacturing an oral solid dosage form which is a tablet comprising a core and a coating over the core, comprising the steps of:
i) Blending a mixture comprising Compound [I] or a pharmaceutically acceptable salt thereof, microcrystalline cellulose, and dicalcium phosphate anhydrous, and optionally a disintegrant and/or a lubricant to form a blend;
ii) Dry granulating the blend;
iii) Compressing the dry granulated blend into a core; and
iii) Applying a coating to the core.

In some embodiments the dry granulation process in step ii) is performed by roller compaction of the blend followed by milling.

In one embodiment there is provided a process for manufacturing an oral solid dosage form which is a tablet comprising a core and a coating over the core, comprising the steps of:
i) Blending a mixture comprising Compound [I] or a pharmaceutically acceptable salt thereof, microcrystalline cellulose, and dicalcium phosphate anhydrous, a disintegrant and a lubricant to form a blend;
ii) Dry granulating the blend;
iii) Blending further lubricant into the dry granulated blend; and
iv) Compressing the blend of step iii) into a core; and
v) Applying a coating to the core.

In some embodiments the dry granulation process in step ii) is performed by roller compaction of the blend followed by milling.

In some embodiments the processes for manufacturing an oral solid dosage form are for manufacturing an oral solid dosage form as described herein.

In some embodiments the oral solid dosage forms as described herein are obtainable by a process for manufacturing an oral solid dosage form described herein.

In embodiments a process for manufacturing an oral solid dosage form may be a continuous direct compression process.

### Medical Uses

By virtue of their ability to inhibit AKT, the pharmaceutical compositions and oral solid dosage forms described in this specification have applications in therapy, for example in the treatment of AKT-mediated disease, such as cancer.

In one embodiment there is provided any pharmaceutical composition or oral solid dosage form described in this specification for use in therapy.

In one embodiment there is provided any pharmaceutical composition or oral solid dosage form described in this specification for use in the treatment of cancer.

The terms “treat,” “treating,” and “treatment” refer to at least partially alleviating, inhibiting, preventing and/or ameliorating a condition, disorder, or disease, such as cancer. The term “treatment of cancer” includes both in-vitro and in-vivo treatments, including in warm-blooded animals such as humans. The effectiveness of treatment of cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival. Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

In one embodiment there is provided the use of any pharmaceutical composition or oral solid dosage form described in this specification for the preparation of a medicament for use in the treatment of cancer.

In one embodiment there is provided a method of treating cancer in a patient (for example a human patient) in need of such treatment, comprising administering to the patient a therapeutically effective amount of any pharmaceutical composition or oral solid dosage form described in this specification.

The term “therapeutically effective amount” refers to that amount of a compound or combination of compounds as described herein that is sufficient to effect the intended application including, but not limited to, disease treatment. A therapeutically effective amount may vary depending upon the intended application (in vitro or in-vivo), or the subject and disease condition being treated (e.g., the weight, age and gender of the subject), the severity of the disease condition, the manner of administration, etc. which can readily be determined by a person skilled in the art. The term also applies to a dose that will induce a particular response in target cells (e.g. the amount of apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

Besides being useful for the treatment of human patients, compositions of the specification, may also be useful for veterinary treatment of companion animals, exotic animals and farm animals, including mammals, rodents, and the like. Such animals include horses, dogs, and cats.

In some embodiments the cancer is selected from breast cancer and prostate cancer.

In some embodiments the cancer is breast cancer.

In some embodiments the cancer is hormone-receptor positive (HR+), HER2-negative breast cancer.

In one embodiment, the cancer is histologically confirmed HR+/HER2− breast cancer determined from the most recent tumour sample (primary or metastatic) per the American Society of Clinical Oncology and College of American Pathologists guideline (Hammond M E H et al., J Clin Oncol 2010, 28(16), 2784-95; Erratum in: J Clin Oncol. 2010, 28(21), 3543; Wolff A C et al., Arch Pathol Lab Med 2018, 142(11), 1364-82). To fulfil the requirement of HR+ disease, a breast cancer must express estrogen receptor (ER) with or without co-expression of progesterone receptor. Therefore, tumours must be:
(a) Estrogen receptor positive (ER+) defined as ≥1% of tumour cells stain positive for ER on immunohistochemistry (IHC) or, if no percentage is available, then an Allred IHC score of ≥3/8,
(b) Progesterone receptor positive defined as 1% of tumour cells stain positive for progesterone receptor on IHC or, if no percentage is available, then an Allred IHC score of ≥3/8; or progesterone receptor negative defined as <1% of tumour cells stain positive for progesterone receptor on IHC or, if no percentage is available, then an Allred IHC score of ≤2/8; or progesterone receptor unknown; and
(c) HER2− defined as 0 or 1+ intensity on IHC, or 2+ intensity on IHC and no evidence of amplification on in situ hybridisation (ISH).

In some embodiments the cancer is locally advanced or metastatic HR+, HER2-negative breast cancer.

In some embodiments the cancer is endocrine-resistant HR+, HER2-negative breast cancer. Endocrine-resistant HR+, HER2-negative breast cancer occurs when the cancer in question has ceased to respond to endocrine treatment (for example a selective estrogen receptor modulator such as tamoxifen, and/or an aromatase inhibitor such as anastrozole or letrozole).

In some embodiments the cancer is metastatic hormone sensitive prostate cancer.

In some embodiments cancer the cancer is metastatic castration resistant prostate cancer.

Granular characterisation of the indicated cancers is detailed in trials referring to capivasertib, AZD5363 and/or (S)-4-amino-N-(1-(4-chlorophenyl)-3-hydroxypropyl)-1-(7H-pyrrolo[2,3-d]pyrimidin-4-yl)piperidine-4-carboxamide on clinicaltrials.gov [for example for example trials NCT03997123, NCT04305496, NCT04862663, NCT05348577 or NCT04493853], nih.gov, clinicaltrialsregister.eu or similar public clinical trial repositories.

In one embodiment there is provided any pharmaceutical composition or oral solid dosage form described in this specification for use in the treatment of cancer, where the pharmaceutical composition or oral solid dosage form is administered in combination with an additional anti-cancer agent.

In one embodiment there is provided a method of treating cancer in an animal patient in need of such treatment, comprising administering to the animal patient a first amount of any pharmaceutical composition or oral solid dosage form described in this specification and a second amount of an additional anti-cancer agent, where the first amount and the second amount together comprise a therapeutically effective amount.

The phrase “in combination with” and similar terms encompass administration of two or more active pharmaceutical ingredients to a subject (for example a patient) and include simultaneous administration in separate compositions, administration at different times in separate compositions, or administration in a composition in which two or more active pharmaceutical ingredients are present.

In embodiments, the administration of a pharmaceutical composition or oral solid dosage form and additional anti-cancer agent is separate, sequential, or simultaneous.

In embodiments, the administration of a pharmaceutical composition or oral solid dosage form and additional anti-cancer agent is separate.

In embodiments, the administration of a pharmaceutical composition or oral solid dosage form and additional anti-cancer agent is sequential.

In embodiments, the administration of a pharmaceutical composition or oral solid dosage form and additional anti-cancer agent is simultaneous.

In one embodiment there is provided any pharmaceutical composition or oral solid dosage form described in this specification for use in the treatment of cancer, where the cancer is metastatic triple negative breast cancer, and the pharmaceutical composition or oral solid dosage form is administered in combination with chemotherapy (for example paclitaxel [e.g. Taxol®] or docetaxel [e.g. Taxotere®]).

In one embodiment there is provided any pharmaceutical composition or oral solid dosage form described in this specification for use in the treatment of cancer, where the cancer is metastatic triple negative breast cancer, and the pharmaceutical composition or oral solid dosage form is administered in combination with paclitaxel.

In one embodiment there is provided any pharmaceutical composition or oral solid dosage form described in this specification for use in the treatment of cancer, where the cancer is endocrine-resistant HR+, HER2-negative breast cancer, and the pharmaceutical composition or oral solid dosage form is administered in combination with a selective estrogen receptor degrader (“SERD”, for example fulvestrant [e.g. Faslodex®] or a pharmaceutically acceptable salt thereof, giredestrant or a pharmaceutically acceptable salt thereof, elacestrant or a pharmaceutically acceptable salt thereof, imlunestrant or a pharmaceutically acceptable salt thereof or camizestrant or a pharmaceutically acceptable salt thereof).

In one embodiment there is provided any pharmaceutical composition or oral solid dosage form described in this specification for use in the treatment of cancer, where the cancer is endocrine-resistant HR+, HER2-negative breast cancer, and the pharmaceutical composition or oral solid dosage form is administered in combination with fulvestrant or a pharmaceutically acceptable salt thereof.

In one embodiment there is provided any pharmaceutical composition or oral solid dosage form described in this specification for use in the treatment of cancer, where the cancer is endocrine-resistant HR+, HER2-negative breast cancer, and the pharmaceutical composition or oral solid dosage form is administered in combination with a SERD (for example fulvestrant or a pharmaceutically acceptable salt thereof, giredestrant or a pharmaceutically acceptable salt thereof, elacestrant or a pharmaceutically acceptable salt thereof, imlunestrant or a pharmaceutically acceptable salt thereof or camizestrant or a pharmaceutically acceptable salt thereof) and a CDK4/6 inhibitor (for example palbociclib [e.g. Ibrance®] or a pharmaceutically acceptable salt thereof, ribociclib [e.g. Kisqali®] or a pharmaceutically acceptable salt thereof and/or abemaciclib [e.g. Verzenois®] or a pharmaceutically acceptable salt thereof.

In one embodiment there is provided any pharmaceutical composition or oral solid dosage form described in this specification for use in the treatment of cancer, where the cancer is endocrine-resistant HR+, HER2-negative breast cancer, and the pharmaceutical composition or oral solid dosage form is administered in combination with fulvestrant or a pharmaceutically acceptable salt thereof and palbociclib or a pharmaceutically acceptable salt thereof.

In one embodiment there is provided any pharmaceutical composition or oral solid dosage form described in this specification for use in the treatment of cancer, where the cancer is endocrine-resistant HR+, HER2-negative breast cancer, and the pharmaceutical composition or oral solid dosage form is administered in combination with fulvestrant or a pharmaceutically acceptable salt thereof and ribociclib or a pharmaceutically acceptable salt thereof.

In one embodiment there is provided any pharmaceutical composition or oral solid dosage form described in this specification for use in the treatment of cancer, where the cancer is metastatic hormone sensitive prostate cancer, and the pharmaceutical composition or oral solid dosage form is administered in combination with abiraterone or a pharmaceutically acceptable salt thereof [e.g. Zytiga®].

In one embodiment there is provided any pharmaceutical composition or oral solid dosage form described in this specification for use in the treatment of cancer, where the cancer is metastatic castration resistant prostate cancer, and the pharmaceutical composition or oral solid dosage form is administered in combination with docetaxel.

In embodiments any pharmaceutical composition or oral solid dosage form described in this specification may be administered in accordance with any published capivasertib clinical trial protocol (for example with respect to patient characteristics, dosage amount and/or dosage frequency), for example any clinical trial protocol referring to capivasertib, AZD5363 and/or (S)-4-amino-N-(1-(4-chlorophenyl)-3-hydroxypropyl)-1-(7H-pyrrolo[2,3-d]pyrimidin-4-yl)piperidine-4-carboxamide published on clinicaltrials.gov, nih.gov, clinicaltrialsregister.eu or similar public clinical trial repositories, for example trials NCT03997123, NCT04305496, NCT04862663, NCT05348577 and/or NCT04493853.

The pharmaceutical compositions and oral solid dosage forms described in this specification may also be utilised for diagnostics and as research tools. For example, pharmaceutical compositions containing compound [I], either alone or in combination with other compounds, can be used as tools in differential and/or combinatorial analyses to elucidate expression patterns of genes expressed within cells and tissues.

### Kits

In one embodiment there is provided a kit comprising any pharmaceutical composition or oral solid dosage form described in this specification and, optionally, instructions teaching the use of the pharmaceutical composition or oral solid dosage form according to the various medical indications described herein.

In embodiments kits may also include other supplemental information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in-vivo models and studies based on human clinical trials. Kits described herein can be provided, marketed and/or promoted to health providers, including physicians, nurses, pharmacists, formulary officials, and the like.

In embodiments kits may be marketed directly to the consumer.

### EXAMPLES

Compound [I] may be prepared according to methods disclosed in WO2009/047563. Compound [I] was used in crystalline Form B. Crystalline Form B is disclosed in WO2013/156772.

DCPA, known also as dicalcium phosphate anhydrous, dibasic calcium phosphate anhydrous and anhydrous calcium hydrogen phosphate, is commercially available from a range of suppliers. Suitable free flowing grades of DCPA for use in the pharmaceutical compositions according to the specification include EMCOMPRESS® from JRS Pharma (www.jrspharma.com) and A-TAB from Innophos (www.innophos.com).

MCC, microcrystalline cellulose, is commercially available from a range of suppliers. Suitable free flowing and high density grades of MCC for use in the pharmaceutical compositions according to the specification include Avicel® pH 102, Avicel® pH 101, Avicel® pH 200 (all from Dupont Pharma, www.dupont.co.uk), VIVAPUR® 102 and VIVAPUR® 200 (from JRS PHARMA GmbH & Co. KG, Rosenberg, Germany).

Suitable commercial grades of magnesium stearate for use in the pharmaceutical compositions according to the specification include LIGAMED® MF from Peter Greven (Peter Greven GmbH & Co. KG, www.peter-greven.de).

Flow function coefficients [FFC] for different pharmaceutical composition blends was determined using a Schultz RST-XS ring shear tester (http://www.dietmar-schulze.com/rstxse.html) at normal stresses and at pre-shear stresses of 1000, 2000 and 4000 Pa.

Particle size was measured by laser diffraction on dry powder.

For tensile strength analysis a Sotax HT100 (www.sotax.com) was used to determine the weight, hardness, thickness and diameter of 10 tablets produced by roller compaction. The tensile strength was calculated from the hardness data and tablets dimensions generated from the Sotax HT100 and the compression tools dimensions using Pitt's equation (see: K. G. Pitt & M. G. Heasley, “Powder Technology”, 2013 [238]p 169-175).

Disintegration was measured using the pharmacopeial disintegration test.

Dissolution experiments were carried out using apparatus II (paddle) dissolution at a temperature of 37 °C. The test was performed using 900 mL of pH 1.2 solution at a paddle speed of 50 rpm, and at pH 6.8 with 0.4% tween at paddle speed of 75 rpm.

Friability studies were performed using the compendial friability tester.

After formulation development work, a number of prototype formulations comprising Compound [I] along with various diluents, disintegrants and lubricants were prepared by a dry granulation process for assessment.

Early development work generated suitable clinical formulations such as that described in Table 1 (Reference Example 1), but although the resultant tablets were suitable for clinical work, they were found to lack ideal properties for tablet manufacture. Further studies to improve the formulations were therefore carried out.

TABLE 1
Early Clinical Tablet Formulation

| | Quantity | | Quantity | | |
|---|---|---|---|---|---|
| | (mg per unit) | | (w/w %)^{a} | | |
| | 80 mg | 200 mg | 80 mg | 200 mg | |
| Composition | Tablet | Tablet | Tablet | Tablet | Function |

Tablet Core

| | | | | | |
|---|---|---|---|---|---|
| Capivasertib | 80.00 | 200.00 | 60.15 | 60.10 | Drug |
| | | | | | substance |
| Microcrystalline | 34.51 | 86.27 | 25.95 | 25.95 | Diluent |
| cellulose | | | | | |
| Mannitol | 11.51 | 28.77 | 8.65 | 8.64 | Diluent |
| Croscarmellose sodium | 5.33 | 13.33 | 4.00 | 4.00 | Disintegrant |
| Magnesium stearate | 2.00 | 5.00 | 1.50 | 1.50 | Lubricant |
| Core tablet weight | 133 | 333 | 133 | 333 | |
| (mg)^{b} | | | | | |

^{a}w/w Percentage quantities of tablet core components are expressed relative to core tablet weight.
^{b}Core tablet weight is rounded to the nearest whole number.

### Example 1: Diluents

Initial studies focused on the amount and nature of the diluent used in the formulations. A range of prototype compositions were investigated using a capivasertib drug loading of 40% or 50% (levels chosen to ensure therapeutic effects in suitable tablet sizes) with diluent mixtures comprising MCC and mannitol or MCC and DCPA in various ratios (Table 2).

TABLE 2
Formulation Parameters Investigated in Initial Screening

| Formulation Factor | Range |
|---|---|
| Capivasertib drug loading | 40% and 50% (w/w) |
| MCC:mannitol ratio | 0:1-1:0 |
| MCC:DCPA ratio | 0:1-1:0 |

Formulations falling within these parameters were judged on a set of preliminary manufacturability criteria including:
1. A tensile strength above 2 MPa to ensure robust tablet cores for further processing, packaging, shipping and handling by patients;
2. A strain rate sensitivity (i.e. sensitivity to tablet press speed) of less than 15% for high commercial speed processing; and
3. A total amount of microcrystalline cellulose (MCC) in the composition not less than 20%, to ensure enough is present to act as a plastic deformation compression aid during roller compaction and tabletting processes.

When analysing representative compositions of Table 2 it was found that a higher ratio of the MCC increases tensile strength (point 1 above) but at the same time increases the strain rate sensitivity (point 2). This is thought to be due to the plastic deformation nature of MCC. DCPA however was found to have the opposite effect, where a higher level decreases the tensile strength and strain rate sensitivity. Based on the predetermined criteria, at 40% drug loading a filler ratio window satisfying both criteria 1 and 2 above was determined to operate between 45:55 to 70:30 of MCC:DCPA. At 50% drug loading, a smaller window of operation was determined between 55:45 to 68:32 of MCC:DCPA.

For those compositions based around a mannitol/MCC diluent mixture it was determined that both 40% and 50% drug loadings left no filler ratio window to operate in, due to high strain rate sensitivity at even the lowest allowable levels of MCC (i.e. at 50% loading and 20% w/w or more MCC it was not possible find an MCC:mannitol ratio that would satisfy both conditions 1 and 2 above). On this basis, an MCC:mannitol combination was considered unfeasible for further development.

Once DCPA had been chosen as secondary diluent, further experiments were carried out to determine suitable drug loading (Table 3).

TABLE 3
Formulation Parameters Investigated in Initial Screening

Quantity (% w/w)/formulation

| | 40% Drug | 50% Drug | 60% Drug |
|---|---|---|---|
| | Loading | Loading | Loading |
| | (Example | (Example | (Example |
| Component | 1a) | 1b) | 1c) |
| Capivasertib | 40.0 | 50.0 | 60.0 |
| MCC^{a} | 32.1 | 26.1 | 20.1 |
| DCPA^{a} | 21.4 | 17.4 | 13.4 |
| Croscarmellose sodium | 5.0 | 5.0 | 5.0 |
| Magnesium stearate | 0.5 | 0.5 | 0.5 |
| (intra-granular) | | | |
| Magnesium stearate | 1.0 | 1.0 | 1.0 |
| (extra-granular) | | | |

^{a}The ratio of MCC to DCPA in all formulations is fixed at 6:4.

All formulation variants 1a-1c were manufactured using the same manufacturing process parameters. The dry powdered ingredients (except the lubricant [magnesium stearate]) were charged to a blender and mixed. The powder was blended with intra-granular lubricant, granulated in a roller compactor and the ribbons milled. The extra-granular lubricant was added to the granules and blended before compressing into tablet cores to obtain 200 mg strength at varied compression pressures and speeds. The compositions were characterised for powder flow (determined using a Schultz RST-XS ring shear tester [http://www.dietmar-schulze.com/rstxse.html] at normal stresses at pre-shear stresses of 1000, 2000 and 4000 Pa), disintegration time at different compression force, tensile strength at different compression speed, and friability (see Table 4).

TABLE 4
Powder Flow, Disintegration Time, Tensile Strength and Friability
of 40%, 50% and 60% drug loading tablet cores

| | Powder | Dis- | Dis- | Tensile | Tensile | Fri- |
|---|---|---|---|---|---|---|
| | Flow | integration | integration | Strength | Strength | ability |
| Variant | (FFC) | Time (s)^{a} | Time (s)^{b} | (MPa)ª | (MPa)ª | (%) |
| 40% w/w | 6.8 | 13 | 17 | 2.2 | 2.1 | 0.14 |
| Drug | | | | | | |
| Loading | | | | | | |
| (1a) | | | | | | |
| 50% w/w | 5.4 | 14 | 16 | 2.4 | 2.3 | 0.15 |
| Drug | | | | | | |
| Loading | | | | | | |
| (1b) | | | | | | |
| 60% w/w | 4.5 | — | — | — | — | — |
| Drug | | | | | | |
| Loading | | | | | | |
| (1c) | | | | | | |

^{a}Compressed at 200 MPa at a press speed of 50 rpm.
^{b}Compressed at 300 MPa at a press speed of 50 rpm.

These results (and the results of standard dissolution testing) demonstrate that:
1. The 60% drug loading formulation (Example 1c) shows relatively poor powder flow properties compared to the lighter drug loading alternatives;
2. The 40% drug loading formulation (Example 1a) shows improved powder flow properties compared to the 50% drug loading formulation;
3. The 40% and 50% formulation variants achieve the target tensile strength at 50 rpm and 90 rpm press speed suggesting that the compositions are robust enough for large/commercial scale production;
4. The 40% and 50% formulation variants show quick disintegration of 17 seconds or less with no significant impact of the applied compression pressure, indicating an effective disintegration mechanism; and
5. The 40% and 50% formulation variants show equivalent immediate release dissolution profiles in low and high pH media.

Based on the differences in flow properties between the drug loading formulation variants, the 40% w/w composition (Example 1a) is better at ensuring a robust flow of powder into the roller compactor, and hence confers manufacturing advantages.

Further experiments were used to determine the influence of MCC:DCPA ratio (3:2 vs 3:1) on strain rate sensitivity (SRS). Two formulations were compared for the study (Table 5).

TABLE 5
3:1 and 3:2 MCC:DCPA ratio Formulations

| | Quantity (% w/w)/formulation | |
|---|---|---|
| | 50% Drug Loading, 3:1 | 50% Drug Loading, 3:2 |
| | MCC:DCPA ratio | MCC:DCPA ratio |
| Component | (Example 1d) | (Example 1b) |
| Capivasertib | 40.0 | 50.0 |
| MCC | 32.6 | 26.1 |
| DCPA | 10.9 | 17.4 |
| Croscarmellose | 5.0 | 5.0 |
| sodium | | |
| Magnesium | 0.5 | 0.5 |
| stearate (intra- | | |
| granular) | | |
| Magnesium | 1.0 | 1.0 |
| stearate (extra- | | |
| granular) | | |

A Sotax HT100 (www.sotax.com) was used to determine the weight, hardness, thickness and diameter of 10 tablets produced from the formulations via roller compaction. The envelope density, E_{D}, was also determined using the information from the tabletting tools, and from the tablet weight and the tablet dimensions using a Sotax HT100 tablet testing system of 10 tablets. The true density, T_{D}, was the weighted true density of the formulation components. Tablet porosity was then calculated as per the Equation Tₚ=100(1−E_{D}/T_{D}), where Tₚ=Tablet Porosity, E_{D}=Envelope Density and T_{D}=True Density.

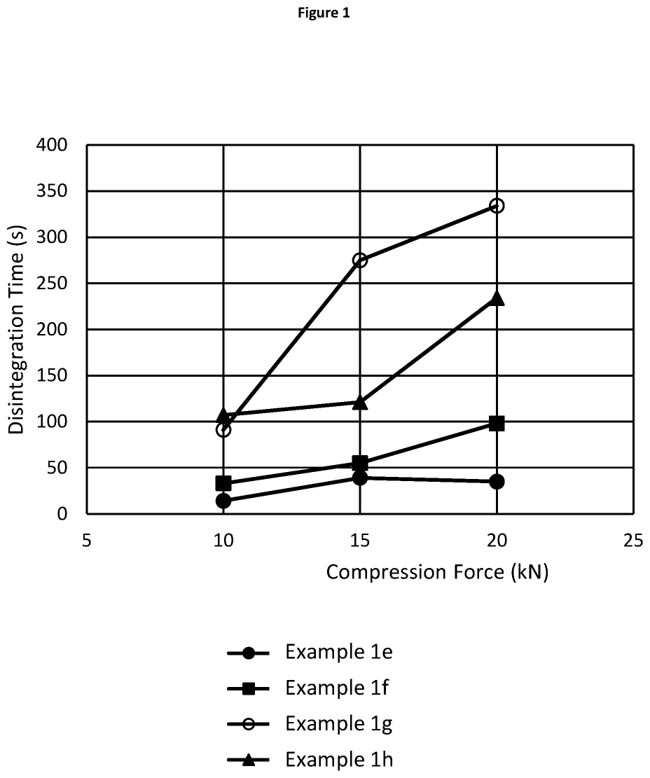

The characterisation results are shown in FIG. 1, and demonstrate that:
1. Compression speed has less impact on tensile strength for the 3:2 (MCC:DCPA) formulation;
2. Increasing the proportion of DCPA reduces SRS; and
3. Tablets with higher DCPA content showed higher porosity at a given tensile strength.

A 3:2 MCC:DCPA ratio therefore has advantages over the 3:1 MCC:DCPA ratio with respect to reproducibility, reduced SRS and higher porosity.

Finally, certain experiments were carried out to compare the disintegration of MCC/mannitol and MCC/DCPA based formulations. Four formulations (Examples 1d, 1e, 1f and 1g, components summarised in Table 6) were prepared by the following methods: A 100 g batch, wet granulation process was used. API and excipients were dry mixed together, and water (2 ml/min) was added during high shear mixing, using a MiPro granulator (0.5 L). Impellor speed was set at 400 rpm and the chopper set at 1500 rpm. The resultant granules were dried using a Vector Fluid bed dryer at 80 °C to <2% w/w moisture, and manually milled using a 1 mm sieve.

Tablets were then compressed according to the following process: Granules were lubricated using a Turbula T2C for 4 minutes at 30 rpm, and compressed into 250 mg tablets using an instrumented F press and an 8 mm normal concave punch. Batches of tablets were prepared by compression to approximately 10 kN (187 MPa), 15 kN (281 Mpa) and 20 kN (375 Mpa).

TABLE 6
MCC:Mannitol vs MCC:DCPA Formulations used in Disintegration Studies

| | Quantity (% w/w)/formulation | | | |
|---|---|---|---|---|
| | MCC/DCPA | MCC/DCPA | MCC/mannitol | MCC/mannitol |
| Component | (Example 1e) | (Example 1f) | (Example 1g) | (Example 1h) |
| Capivasertib | 15.0 | 15.0 | 15.0 | 15.0 |
| MCC | 49.0 | 49.0 | 49.0 | 49.0 |
| DCPA | 25.0 | 25.0 | 0 | 0 |
| Mannitol | 0 | 0 | 25.0 | 25.0 |
| Sodium Starch Glycolate | 5.0 | 0 | 5.0 | 0 |
| Croscarmellose sodium | 0 | 5.0 | 0 | 5.0 |
| Hydroxypropyl cellulose | 4.0 | 4.0 | 4.0 | 4.0 |
| Sodium stearyl fumarate | 1.5 | 0 | 0 | 1.5 |
| Magnesium stearate | 0 | 1.5 | 1.5 | 0 |
| Colloidal silicon dioxide | 0.5 | 0.5 | 0.5 | 0.5 |

Once tablets of each formulation were prepared at varying compression conditions, they were tested to see their disintegration using the standard pharmacopeial test. The results are shown in FIG. 1, which illustrates disintegration time for Examples 1e, 1f, 1g and 1h prepared with varying compression force. It can be seen that the MCC/DCPA formulations 1e and 1f exhibited faster disintegration times compared to MCC/mannitol formulations 1g and 1h.

### Example 2: Disintegrants

Croscarmellose sodium was selected for study as preliminary experiments showed it could confer good stability and disintegration performance. The Examples shown in Table 3 contained croscarmellose sodium (5% w/w) and showed a quick and robust disintegration profile (17 s and 16 s for Examples 1a and 1b respectively, when analysed at pH 1.2 (50 rpm) and pH 6.8 with 0.4% tween [75 rpm]). A confirmatory study was undertaken to explore the sensitivity of the formulation to the disintegrant level. The level of the disintegrant, croscarmellose sodium, was adjusted in the formulation from 5% to 0% w/w to assess the impact on the disintegration and dissolution performance. The level of fillers (but not their ratio) was adjusted to maintain the tablet compression weight (Table 7).

TABLE 7
Disintegrant Variants

Quantity (% w/w)/formulation

| | 5% Disintegrant | 0% Disintegrant |
|---|---|---|
| Component | (Example 1a) | (Example 2) |
| Capivasertib | 40.0 | 50.0 |
| MCC^{a} | 32.1 | 35.1 |
| DCPA^{a} | 21.4 | 23.4 |
| Croscarmellose sodium | 5.0 | 0.0 |
| Magnesium stearate | 0.5 | 0.5 |
| (intra-granular) | | |
| Magnesium stearate | 1.0 | 1.0 |
| (intra-granular) | | |

^{a}The ratio of MCC to DCPA in both formulations is fixed at 6:4.

Both variants were manufactured using the same manufacturing process parameters. The dry powdered ingredients (except the lubricant) were charged to a blender and mixed. The powder was blended with intra-granular lubricant, granulated in a roller compactor and the ribbons milled. The extra-granular lubricant was added to the granules and blended before compressing into tablet cores of 500 mg compression weight.

As shown in Table 8, the disintegration and dissolution is rapid and complete for both Example 1a and Example 2, demonstrating that the formulation is an instant release formulation robust with respect to changes in the disintegrant level.

TABLE 8
Dissolution and disintegration of Examples 1a and 2

| | 5% Disintegrant | 0% Disintegrant |
|---|---|---|
| Variant | (Example 1a) | (Example 2) |
| Disintegration Time (s)^{a} | 23 ± 1 | 23 ± 3 |
| Dissolution (%) at 30 minutes | 97.4 ± 4.3 | 99.6 ± 2.4 |
| at pH 1.2^{b} | | |

^{a}Results are mean ± standard deviation, N = 3.
^{b}Results are mean ± standard deviation, N = 6.

### Example 3: Lubricants

Following initial screens magnesium stearate was selected for further development as it showed good stability in prototype film coated tablets. The level of lubricant was further investigated to assess the impact of higher levels of this hydrophobic and anti-adhesive material on tensile strength and disintegration/dissolution profile of the tablets.

Three variants were produced from the same common granule batch with a consistent level of intra-granular lubricant (0.5% w/w). The extra-granular lubricant was incorporated to produce variants with 0.75, 1.0%, 1.25% and 1.5% w/w, equating to total levels of 1.25, 1.5%, 1.75 and 2.0% w/w respectively (Table 9, Examples 3a-3d respectively).

TABLE 9
Extra-Granular Lubricant Variants

| | Quantity (% w/w)/formulation | | | |
|---|---|---|---|---|
| | 0.75% Extra- | 1.00% Extra- | 1.25% Extra- | 1.50% Extra- |
| | granular | granular | granular | granular |
| | Lubricant | Lubricant | Lubricant | Lubricant |
| Component | (Example 3a) | (Example 3b) | (Example 3c) | (Example 3d) |
| Capivasertib | 40.10 | 40.00 | 39.0 | 38.80 |
| MCC | 32.18 | 32.10 | 32.02 | 31.94 |
| DCPA | 21.45 | 21.40 | 21.35 | 21.29 |
| Croscarmellose sodium | 5.01 | 5.00 | 5.00 | 5.00 |
| Magnesium stearate | 0.50 | 0.50 | 0.50 | 0.50 |
| (intra-granular) | | | | |
| Magnesium stearate | 0.75 | 1.00 | 1.25 | 1.50 |
| (intra-granular) | | | | |

To prepare Examples 3a-3d, the dry powdered ingredients (except the lubricant) were charged to a blender and mixed. The powder was blended with intra-granular lubricant, granulated in a roller compactor and the ribbons milled. The common granules for all the 3 variants were roller compacted at a relatively high roll force of 12 kN/cm as a “worst case” for commercial tablet compression and dissolution. The extra-granular lubricant was then added to the granules and mixed together before compressing into tablet cores with a 500 mg compression weight.

The manufacturability of the different variants was then investigated, generating the results shown in Table 10.

TABLE 10
Extra-Granular Lubricant Variant Properties

| | Extra-granular Lubricant Level^{a} | | | |
|---|---|---|---|---|
| | 0.75% Extra- | 1.00% Extra- | 1.25% Extra- | 1.50% Extra- |
| | granular | granular | granular | granular |
| | Lubricant | Lubricant | Lubricant | Lubricant |
| Assessment Criteria | (Example 3a) | (Example 3b) | (Example 3c) | (Example 3d) |
| Ejection Force (N) | 520 ± 10 | 540 ± 8 | 493 ± 9 | 471 ± 14 |
| Tensile Strength | 2.4 ± 0.1 | 1.7 ± 0.1 | 1.7 ± 0.1 | 1.6 ± 0.1 |
| Disintegration Time (s)^{b} | 22 ± 0 | 22 ± 2 | 25 ± 1 | 26 ± 0 |
| Dissolution (%) at 30 | 97.5 ± 0.5 | 98.5 ± 2.4 | 97.3 ± 1.7 | 97.3 ± 0.4 |
| minutes at pH 1.2^{c} | | | | |

^{a}Granules roller compacted at 12 kN/cm and compressed at 35 kN.
^{b}Results are mean ± standard deviation, N = 3.
^{c}Results are mean ± standard deviation, N = 6.

The following conclusions can be drawn:
1. A small gradual decrease in the ejection force on the tablet press was seen at an extra-granular lubricant level of 1.25% (w/w) or higher. However, no issues related to tablet ejection were seen and therefore these small differences in ejection force are practically negligible;
2. At extra-granular lubricant levels of 1.0% or higher, a step reduction in tensile strength was seen when compared the lowest level investigated of 0.75%. However, all lubricant levels investigated achieved tensile strengths in excess of the 1.5 MPa, which while not as robust as 2 MPa, would still be suitable for commercialisation;
3. Disintegration and dissolution of all the variants were rapid and complete (>85% in 30 minutes), demonstrating that the increase in level of magnesium stearate has no impact on the in-vitro performance of the tablets.

The intra-granular lubricant level was also investigated. Two variants (Examples 3e and 3f) were produced at 0.5% and 1.0% intra-granular lubricant with a consistent level of extra-granular lubricant level (1.0%). The intra-granular lubricant was incorporated to produce variants with 0.5% and 1.0% w/w, equating to total levels of 1.5% and 2.0% w/w respectively (Table 11).

TABLE 11
Intra-Granular Lubricant Variants

| | Quantity (% w/w)/formulation | |
|---|---|---|
| | 0.50% Intra- | 1.00% Intra- |
| | granular Lubricant | granular Lubricant |
| Component | (Example 3e) | (Example 3f) |
| Capivasertib | 40.00 | 40.00 |
| MCC | 32.10 | 31.80 |
| DCPA | 21.40 | 21.20 |
| Croscarmellose sodium | 5.00 | 5.00 |
| Magnesium stearate | 0.50 | 1.00 |
| (intra-granular) | | |
| Magnesium stearate | 1.00 | 1.00 |
| (extra-granular) | | |

To prepare Examples 3e and 3f, the dry powdered ingredients (except the lubricant) were charged to a blender and mixed. The powder was blended with intra-granular lubricant, granulated in a roller compactor and the ribbons milled. The extra-granular lubricant was added to the granules and mixed together before compressing into tablet cores with a 500 mg compression weight.

The manufacturability of Examples 3e and 3f was then investigated, generating the results shown in Table 12.

TABLE 12
Intra-Granular Lubricant Variant Properties

| | Intra-granular Lubricant Level^{a} | |
|---|---|---|
| | 0.50% Intra- | 1.00% Intra- |
| | granular Lubricant | granular Lubricant |
| Assessment Criteria | (Example 3e) | (Example 3f) |
| Ejection Force (N) | 459 ± 10 | 462 ± 9 |
| Tensile Strength | 1.6 ± 0.1 | 1.5 ± 0.1 |
| Disintegration Time (s)^{b} | 23 ± 1 | 26 ± 0 |

^{a}Granules roller compacted at 12 kN/cm and compressed at 35 kN.
^{b}Results are mean ± standard deviation, N = 3.

The following conclusions can be drawn:
1. For both intra-granular lubricant levels investigated, tensile strengths in excess of 1.5 MPa were achieved. The data demonstrate that varying the lubricant level within these ranges has negligible effect on the tensile strength of the resultant tablets; and
2. Disintegration of 0.5% and 1.0% w/w magnesium stearate variants was quick (<1 minute), demonstrating that the increase in level of Intra-granular lubricant has no impact on the disintegration performance of the tablet.

### Example 4: Particle Size

A range of drug substance particle size was investigated to assess the impact on the quality attributes and manufacturability of drug product. Three particle size variants were generated by milling the dug substance to 3 different target D(v,0.9) particle sizes of approximately 70 μm, 100 μm and 170 μm. All particle size variants were manufactured using the same formulation and manufacturing process parameters. The dry powdered ingredients (except the lubricant) were charged to a blender and mixed. The powder was then blended with intra-granular lubricant, granulated in a roller compactor and the ribbons milled. The extra-granular lubricant was added to the granules and blended before compressing into tablet cores of 500 mg compression weight. The roll force and compression force were varied to assess for potential interactive effect with drug substance particle size.

TABLE 13
Characterisation of drug substance particle size variants

| | D(v, 0.9) of particle size variants (μm)^{a} | | |
|---|---|---|---|
| Assessment criteria | 70 | 100 | 170 |
| Neat drug substance | 2.5 | 3.6 | 2.4 |
| FFC | | | |
| Blend FFC at 4000 MPa | 4.7 | 6.4 | 5.6 |
| pre-shear | | | |
| Granules FFC4000 | 8.1 | 8.3 | 10.5 |
| MPa pre-shear | | | |
| Tensile strength (MPa) | 2.3 ± 0.1 | 2.1 ± 0.1 | 2.1 ± 0.1 |
| Disintegration time | 22 ± 2 | 16 ± 2 | 23 ± 1 |
| (seconds)^{b} | | | |
| Dissolution (%) at 30 | 99.8 ± 2.1 | 97.8 ± 0.7 | 95.9 ± 2.0 |
| minutes in pH 1.2^{c} | | | |

^{a}Granules roller compacted at 12 kN/cm and compressed at 35 kN.
^{b}Results are mean ± standard deviation, N = 3.
^{c}Results are mean ± standard deviation, N = 6.

The following conclusions can be drawn:
1. For all particle size variants, the flow property of the neat drug substance is poor (FFC 85% in 30 minutes);
2. The poor and variable flow property of neat drug substance for all particle size variants is improved categorically from poor to easy flowing for the dry blend;
3. The roller compaction process further improves the flow property to free flowing for the granules and reduces the variability between drug substance particle size variants;
4. For all particle size variants, tensile strengths in excess of 2 MPa were achieved;
5. The data demonstrate that varying the particle size of drug substance has no impact on the tensile strength of the resultant tablets (and that using the formulation blends mean that good flow properties can be achieved regardless of particle size); and
6. Disintegration and dissolution of all the variants were rapid and complete (>85% in 30 minutes), demonstrating that the particle size of drug substance has negligible impact on the in vitro performance of the tablet within the investigated range.

### Example 5: Coatings

Two investigations were performed to select a suitable coating system. The first study investigated Opadry® I (HPMC based) and Opadry® II (PVA based) systems where debossed capivasertib tablets were assessed for their appearance, dissolution and physical properties after coating with the different systems. Opadry® II was selected as it resulted in superior appearance quality of the debossing. The second study investigated the scuffing issue seen on Opadry® II at large scale manufacturing where few coating systems and process parameters were assessed. Aquarius Preferred was selected as it showed lower risk of scuffing while maintaining the appearance quality of Opadry® II system. The composition of the selected coating system is shown in Table 14.

TABLE 14
Composition of Aquarius Preferred Beige (BPP315509)

| Ingredient | Quantitative Composition (% w/w) |
|---|---|
| Hypromellose 2910 | 25.000 |
| Copovidone plasdone S630 | 22.500 |
| Polyethylene glycol 3350 | 9.500 |
| Polydextrose | 15.000 |
| Medium chain triglycerides | 3.000 |
| Titanium dioxide | 22.080 |
| Iron oxide yellow | 2.340 |
| Iron oxide red | 0.410 |
| Iron oxide black | 0.170 |

A typical level of coating for immediate release tablet of 0.067 mg/mm² was selected the final formulations, equating to 4.0% and 3.8% weight gain for 160 and 200 mg tablet strengths respectively. A confirmatory study was undertaken to ensure that the level of tablet coat does not have a significant impact on the product quality attributes of appearance and dissolution. Tablets were coated using a perforated drum coater and removed at 8 intervals throughout the coating process to achieve a range of tablet weight gains (1.1-7.2% w/w). All 8 samples taken with the range passed the criteria for coat uniformity and completeness without any minor or major physical defects.

The target and the extreme weight gain variants from the samples (target 3.8% and highest 7.2%) were also assessed for dissolution (Table 15). As anticipated from a non-functional film-coat, the level of tablet coat does not have a significant impact on release.

TABLE 15
Characterisation of coating weight gain variants

| | Coating Weight Gain Variants (% w/w gain) | |
|---|---|---|
| | 3.8% Weight Gain | 7.2% Weight Gain |
| Assessment Criteria | (Example 5a) | (Example 5b) |
| Dissolution (%) at 30 | 98.8 ± 0.7 | 98.9 ± 1.1 |
| minutes at pH 1.2^{a} | | |

^{a}Results are mean ± standard deviation, N = 6.

### Example 6: Final Capivasertib Tablets

Following the studies above, tablets with the components listed in Table 16 were selected as a suitable commercial formulation.

TABLE 16
Final Capivasertib Tablet Formulations

| | Quantity | | Quantity | | |
|---|---|---|---|---|---|
| | (mg per unit) | | (w/w %)^{a} | | |
| | 160 mg | 200 mg | 160 mg | 200 mg | |
| | Tablet | Tablet | Tablet | Tablet | |
| Composition | (Example 6a) | (Example 6b) | (Example 6a) | (Example 6b) | Function |

Tablet Core

| | | | | | |
|---|---|---|---|---|---|
| Capivasertib | 160.0 | 200.00 | 40.00 | 40.00 | Drug |
| | | | | | substance |
| Microcrystalline | 128.4 | 160.5 | 32.10 | 32.10 | Diluent |
| cellulose | | | | | |
| Dibasic calcium | 85.6 | 107.0 | 21.40 | 21.40 | Diluent |
| phosphate anhydrous | | | | | |
| (DCPA) | | | | | |
| Croscarmellose sodium | 20.0 | 25.0 | 5.00 | 5.00 | Disintegrant |
| Magnesium stearate | 6.0 | 7.5 | 1.50 | 1.50 | Lubricant |
| Core tablet weight | 400 | 500 | 400 | 500 | |
| (mg)^{b} | | | | | |

Tablet Coating^{c}

| | | | | | |
|---|---|---|---|---|---|
| Hypromellose 2910 | 4.00 | 4.79 | 25.000 | 25.000 | Film-former |
| Copovidone plasdone | 3.60 | 4.31 | 22.500 | 22.500 | Film-former |
| S630 | | | | | |
| Polyethylene glycol | 1.52 | 1.82 | 9.500 | 9.500 | Plasticiser |
| 3350 | | | | | |
| Polydextrose | 2.40 | 2.87 | 15.000 | 15.000 | Film-former |
| caprylic/capric | 0.48 | 0.57 | 3.000 | 3.000 | Film-former |
| triglycerides | | | | | |
| Titanium dioxide | 3.53 | 4.23 | 22.060 | 22.263 | Opacifier |
| Iron oxide yellow | 0.37 | 0.45 | 2.310 | 2.368 | Colouring |
| | | | | | agent |
| Iron oxide red | 0.07 | 0.08 | 0.438 | 0.421 | Colouring |
| | | | | | agent |
| Iron oxide black | 0.03 | 0.03 | 0.188 | 0.158 | Colouring |
| | | | | | agent |
| Purified water^{d} | qs | qs | | | Solvent |
| Nominal coated tablet | 416 | 519 | 416 | 519 | |
| weight (mg) | | | | | |

^{a}w/w Percentage quantities of tablet core components are expressed relative to core tablet weight, w/w percentage quantities of coating components are expressed relative to nominal coating weight.
^{b}Core tablet weight is rounded to the nearest whole number.
^{c}Target amount corresponding to 4% of the core tablet weight for the 160 mg tablet and 3.8% of the core tablet weight for the 200 mg tablet.
^{d}Purified water is used as a solvent/carrier during the film coating process and is ultimately removed during the process.

The stability of the tablets shown in Table 16 throughout their typical shelf life has been confirmed through standard long term stability studies. Tablets stored under different ambient conditions (humidity, temperature etc.) in HDPE bottles were monitored for changes in tablet quality attributes (such as description, assay, organic impurities, chiral purity, dissolution, water content, microbial content) for up to 5 years. The available data on capivasertib tablets 160 mg and 200 mg (i.e. Examples 6a and 6b respectively) demonstrates that there are no significant changes to the tablets in the long term or under accelerated storage conditions. The dissolution rate of the tablets slows with age irrespective of storage environment, however not significantly as all batches satisfied specification after 5 years storage at long term conditions (25 °C/60% RH and 30 °C/75% RH). The data supports a shelf-life for capivasertib 160 mg and 200 mg tablets of 5 years when stored below 30 °C in HDPE bottles.

As the data elsewhere in this specification shows, the tablets described in Table 16 have good dissolution profiles, enabling their use as an immediate release therapy. They are also have favourable physical properties (for example tensile strength. strain rate sensitivity and an ability to aid compression) that improve tablet manufacturability, for instance with respect to powder flow to the roller compactor after dry granulation and punch filming. Finally, the appearance of the tablet appearance after coating is improved compared to earlier formulations.

1. A pharmaceutical composition comprising Compound [I] or a pharmaceutically acceptable salt thereof, microcrystalline cellulose and dicalcium phosphate anhydrous.

2. A pharmaceutical composition as claimed in claim 1, comprising Compound [I], microcrystalline cellulose and dicalcium phosphate anhydrous.

3. A pharmaceutical composition as claimed in claim 1 or claim 2, which is an immediate release pharmaceutical composition.

4. A pharmaceutical composition as claimed in any of the preceding claims, where the pharmaceutical composition comprises up to 65% w/w of Compound [I] or a pharmaceutically acceptable salt thereof.

5. A pharmaceutical composition as claimed in any of the preceding claims, where the pharmaceutical composition comprises 40%-50% w/w of Compound [I] or a pharmaceutically acceptable salt thereof.

6. A pharmaceutical composition as claimed in any of the preceding claims, where the pharmaceutical composition comprises about 50% w/w of Compound [I] or a pharmaceutically acceptable salt thereof.

7. A pharmaceutical composition as claimed in any of the claims 1-5, where the pharmaceutical composition comprises about 40% w/w of Compound [I] or a pharmaceutically acceptable salt thereof.

8. A pharmaceutical composition as claimed in any of the preceding claims, where the ratio of microcrystalline cellulose to dicalcium phosphate anhydrous is from 45:55 to 70:30.

9. A pharmaceutical composition as claimed in claim 8, where the ratio of microcrystalline cellulose to dicalcium phosphate anhydrous is from 55:45 to 68:32.

10. A pharmaceutical composition as claimed in claim 8 or claim 9, where the ratio of microcrystalline cellulose to dicalcium phosphate anhydrous is about 6:4.

11. A pharmaceutical composition as claimed in any of the preceding claims, where the combined amount of microcrystalline cellulose and dicalcium phosphate anhydrous is from 15% w/w to 65% w/w.

12. A pharmaceutical composition as claimed in claim 11, where the combined amount of microcrystalline cellulose and dicalcium phosphate anhydrous is from 30% w/w to 65% w/w.

13. A pharmaceutical composition as claimed in claim 12, where the combined amount of microcrystalline cellulose and dicalcium phosphate anhydrous is from 50% w/w to 55% w/w.

14. A pharmaceutical composition as claimed in any of the preceding claims, further comprising at least one disintegrant in an amount of up to 10% w/w.

15. A pharmaceutical composition as claimed in claim 14, where the at least one disintegrant is present in an amount of up to 5% w/w.

16. A pharmaceutical composition as claimed in claim 14 or claim 15, where the at least one disintegrant is croscarmellose sodium.

17. A pharmaceutical composition as claimed in any of the preceding claims, further comprising at least one lubricant in an amount of up to 4% w/w.

18. A pharmaceutical composition as claimed in claim 17, where the at least one lubricant is present in an amount of up to 1.5% w/w.

19. A pharmaceutical composition as claimed in claim 17 or claim 18, where the at least one lubricant is magnesium stearate.

20. A pharmaceutical composition as claimed in claim 1, comprising:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 50%-55% w/w;
Croscarmellose sodium in an amount of about 5% w/w; and
Magnesium stearate in an amount of about 1.5% w/w.

21. An oral solid dosage form comprising a pharmaceutical composition as claimed in any of the preceding claims, where the oral solid dosage form is a capsule.

22. An oral solid dosage form comprising a pharmaceutical composition as claimed in any of claims 1 to 20, where the oral solid dosage form is a tablet.

23. An oral solid dosage form as claimed in claim 22, where the oral solid dosage form is a tablet comprising a core comprising a pharmaceutical composition as claimed in any of claims 1 to 20, and a coating over the core.

24. An oral solid dosage form as claimed in claim 23, wherein the core comprises:
About 40% w/w Compound [I];
microcrystalline cellulose and dicalcium phosphate anhydrous, where the ratio of microcrystalline cellulose to dicalcium phosphate anhydrous is about 6:4, and the combined amount of microcrystalline cellulose and dicalcium phosphate anhydrous is 50%-55% w/w;
Croscarmellose sodium in an amount of about 5% w/w; and
Magnesium stearate in an amount of about 1.5% w/w.

25. An oral solid dosage form as claimed in claim 23 or claim 24, where the coating comprises hypromellose 2910, copovidone plasdone S630, polyethylene glycol 3350, polydextrose, at least one caprylic/capric triglyceride, titanium dioxide, iron oxide yellow, iron oxide red and iron oxide black.

26. An oral solid dosage form as claimed in any of claims 21 to 25, containing 80 mg, 160 mg or 200 mg of Compound [I].

27. An oral solid dosage form as claimed in any of claims 21 to 26, comprising:
200 mg Compound [I];
160.5 mg microcrystalline cellulose;
107.0 mg dicalcium phosphate anhydrous;
25.0 mg croscarmellose sodium; and
7.5 mg magnesium stearate.

28. A process for manufacturing an oral solid dosage form as claimed in any of claims 22 to 25, comprising the steps of:
i) Blending a mixture comprising Compound [I] or a pharmaceutically acceptable salt thereof, microcrystalline cellulose, and dicalcium phosphate anhydrous and optionally a disintegrant and/or a lubricant to form a blend;
ii) Dry granulating the blend; and
iii) Compressing the dry granulated blend into a tablet.

29. A process as claimed in claim 28, where the dry granulation process in step ii) is performed by roller compaction of the blend followed by milling.

30. A pharmaceutical composition as claimed in any of claims 1 to 20, or an oral solid dosage form as claimed in any of claims 21 to 27, for use in therapy.

31. A pharmaceutical composition as claimed in any of claims 1 to 20, or an oral solid dosage form as claimed in any of claims 21 to 27, for use in the treatment of cancer.

32. The use of a pharmaceutical composition as claimed in any of claims 1 to 20, or an oral solid dosage form as claimed in any of claims 21 to 27, for the preparation of a medicament for use in the treatment of cancer.

33. A method of treating cancer in a patient in need of such treatment, comprising administering to the patient a therapeutically effective amount of a pharmaceutical composition as claimed in any of claims 1 to 20, or an oral solid dosage form as claimed in any of claims 21 to 27.
